(19) United States
(12) Patent Application Publication
(10) Pub. No.: US 2015/0250520 A1
(43) Pub. Date: Sep. 10, 2015

(54) SURGICAL INSTRUMENT AND METHOD

(71) Applicant: Warsaw Orthopedic, Inc., Warsaw, IN (US)

(72) Inventors: William A. Rezach, Atoka, TN (US); Nicholas M. Benson, Cordova, TN (US)

(73) Assignee: Warsaw Orthopedic, Inc., Warsaw, IN (US)

(21) Appl. No.: 14/201,286

(22) Filed: Mar. 7, 2014

(51) Int. Cl. A61B 17/88

(52) U.S. Cl. CPC A61B 17/8875

(57) ABSTRACT

A surgical instrument includes a first end including a gripping surface. At least a portion of the gripping surface has a spherical configuration. A second end is connected with the first end such that an instrument is prevented from engaging the first end to separate the first end from the second end. The second end is configured to engage a bone fastener. A distance from the first end to the second end is equal to or less than approximately 22.5 cm. Systems and methods are disclosed.

### TECHNICAL FIELD

The present disclosure generally relates to medical devices for the treatment of musculoskeletal disorders, and more particularly to a surgical system for delivering and/or fastening implants with a surgical site and a method for treating a spine.

### BACKGROUND

Spinal pathologies and disorders such as scoliosis and other curvature abnormalities, kyphosis, degenerative disc disease, disc hemiation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, implants such as bone fasteners, connectors, plates and vertebral rods are often used to provide stability to a treated region. These implants can redirect stresses away from a damaged or defective region while healing takes place to restore proper alignment and generally support the vertebral members. Surgical instruments are employed, for example, to engage the fasteners for attaching rods and plates to the exterior of two or more vertebral members. This disclosure describes an improvement over these prior art technologies.

### SUMMARY

In one embodiment, a surgical instrument is provided. The surgical instrument includes a first end including a gripping surface. At least a portion of the gripping surface has a spherical configuration. A second end is connected with the first end such that an instrument is prevented from engaging the first end to separate the first end from the second end. The second end is configured to engage a bone fastener. A distance from the first end to the second end is equal to or less than approximately 22.5 cm. In some embodiments, systems and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a surgical implant system in accordance with the principles of the present disclosure;

FIG. 2 is a perspective view of the components shown in FIG. 1;

FIG. 3 is a perspective view of components of one embodiment of a surgical implant system in accordance with the principles of the present disclosure;

FIG. 4 is a cross-section view of the components shown in detail A in FIG. 1;

FIG. 5 is a cross-section view of the components shown in detail B in FIG. 1;

FIG. 6 is an end view of a component of the spinal implant system shown in FIG. 1;

FIG. 7 is an end view of a component of the surgical implant system shown in FIG. 1;

FIG. 8 is an end view of a component of the surgical implant system shown in FIG. 1;

FIG. 9 is a cross-section view of components of the surgical implant system shown in FIG. 1; and

FIG. 10 is a cross section view of components of one embodiment of a surgical implant system in accordance with the principles of the present disclosure.

### DETAILED DESCRIPTION

The exemplary embodiments of a surgical system are discussed in terms of medical devices for the treatment of musculoskeletal disorders and more particularly, in terms of a surgical implant system for delivering and/or fastening implants with a surgical site and a method for treating a spine. In one embodiment, the present system includes a surgical instrument that allows the surgeon to place a pedicle screw. In some embodiments, the surgical instrument probes, taps and/or fastens a bone screw. In some embodiments, a surgical instrument is provided that facilitates a procedural flow of a surgical method and streamlines the surgical method.

In one embodiment, the surgical instrument includes a ball shaped handle. In some embodiments, the ball shaped handle includes a metal cap disposed at one end configured for impacting. In some embodiments, the metal cap includes geometry configured for adaptation with a driving handle. In one embodiment, the metal cap geometry includes a hex interface. In one embodiment, the interface may be a geometry that allows for the transfer of torque.

In one embodiment, the surgical instrument includes a collet and sleeve style connection mechanism to ensure a rigid interface between a driver and a screw. In some embodiments, the rigid interface between the driver and the screw may include alternate interface geometries, such as, for example, frictional engagement, threaded engagement, mutual grooves, adhesive, and/or raised element, and/or semi-rigid geometries. In some embodiments, the surgical instrument may be employed with posted screws, pedicle screws, uni-axial screws (UAS), multi-axial screws (MAS), side loading screws, and sagittal angulation screws (SAS).

In one embodiment, the surgical instrument is configured for attachment with one or a plurality of alternately configured and/or dimensioned handles used to drive a screw after a screw trajectory has been established. In one embodiment, ratcheting handles are utilized. In some embodiments, the driving handle is attached using quick connect features or keyed geometry, such as, for example, triangle, hex, square or hexalobe.

In some embodiments the surgical instrument of the present disclosure is employed with a method as part of a streamlined surgical process. In one embodiment, the method includes inserting a bone screw into bone and includes the step of attaching a bone screw to a ball handle driver. In one embodiment, the method includes the step of forming a starter hole with a tip of a bone screw by use of a longitudinal pushing force exerted on the bone screw. In one embodiment, the pushing force is performed by hand or by hitting or impacting an end of the driver. In one embodiment, a pilot hole is then formed by further exerting a longitudinal pushing force to the bone screw. In one embodiment, the bone screw is removed from the pilot hole and the surgeon feels the pedicle walls to ensure that the trajectory is within the pedicle walls. Once confirmed, the bone screw is re-inserted into the pilot hole. In one embodiment, a driving handle is then attached to the ball handle driver to drive the bone screw such that the bone screw is fixedly secured to the bone. In one embodiment, the driver engages the screw such that the screw is fixed relative to the driver. In some embodiments, an outer surface of the screw has a hexagonal configuration for engagement with a tool, such as, for example, a driver that may be used to rotate the screw. For example, the screw is rotated relative to the bone such that a portion of the screw rotates within the pilot hole. As the screw rotates within the pilot hole, threads on an outer surface of the screw engage the bone such that the screw penetrates the bone. This allows the screw to be implanted into the bone in a single step.

In one embodiment, the system includes a surgical instrument configured for disassembly to facilitate cleaning of each of the components of the surgical instrument. This configuration provides access to areas of the surgical instrument, including difficult to reach areas and/or inaccessible areas due to a surgical instruments assembled configuration. In some embodiments, the surgical instrument is configured for disassembly and assembly. In one embodiment, the surgical instrument includes a collet style connection mechanism to facilitate disassembly and assembly. In some embodiments, the surgical instrument may be disassembled and assembled without additional tools or other instruments.

In some embodiments, the system of the present disclosure may be employed to treat spinal disorders such as, for example, degenerative disc disease, disc hemiation, osteoporosis, spondylolisthesis, stenosis, scoliosis and other curvature abnormalities, kyphosis, tumor and fractures. In some embodiments, the system of the present disclosure may be employed with other osteal and bone related applications, including those associated with diagnostics and therapeutics. In some embodiments, the disclosed system may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The system of the present disclosure may also be alternatively employed with procedures for treating the lumbar, cervical, thoracic, sacral and pelvic regions of a spinal column. The system of the present disclosure may also be used on animals, bone models and other non-living substrates, such as, for example, in training, testing and demonstration.

The system of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

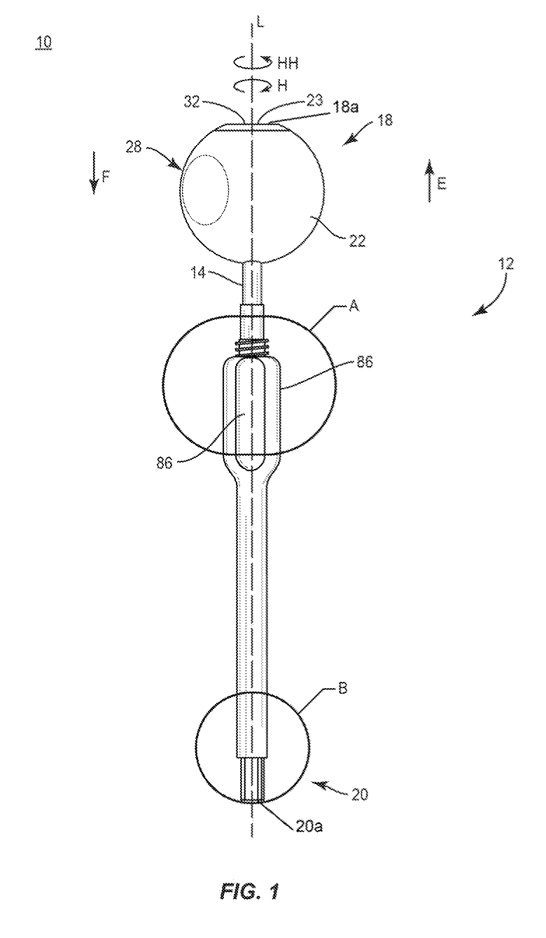

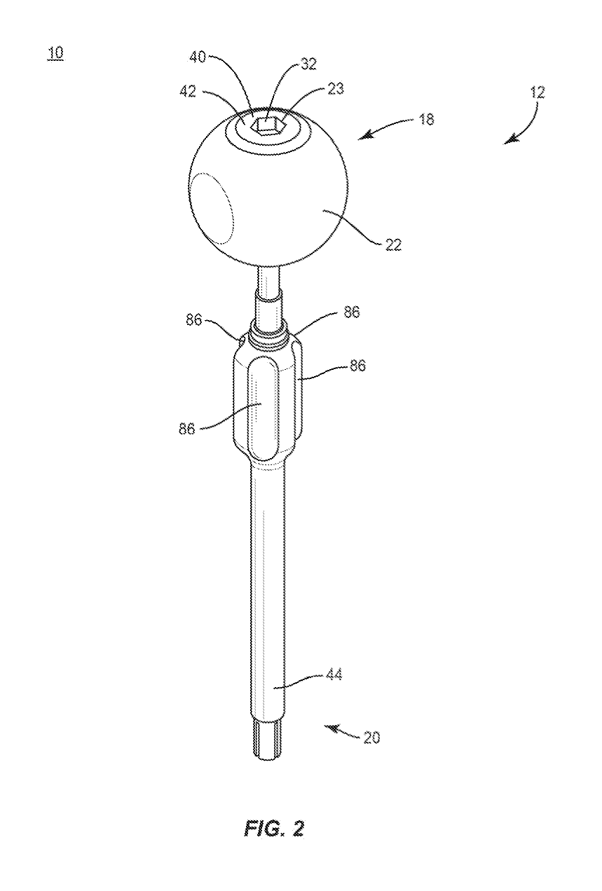

The following discussion includes a description of a surgical system including a surgical instrument, related components and methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-10, there are illustrated components of a surgical implant system 10, in accordance with the principles of the present disclosure.

The components of system 10 can be fabricated from biologically acceptable materials suitable for medical applications, including metals, synthetic polymers, ceramics and bone material and/or their composites. For example, the components of system 10, individually or collectively, can be fabricated from materials such as stainless steel alloys, aluminum, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials. The components of system 10 may be monolithically formed, integrally connected or include fastening elements and/or instruments, as described herein.

System 10, which includes surgical instrument 12 is employed, for example, with an open or mini-open, minimal access and/or minimally invasive including percutaneous surgical technique to deliver and fasten an implant at a surgical site within a body of a patient, for example, a section of a spine. In one embodiment, the components of system 10 are configured to fix a bone fastener with tissue for a surgical treatment to treat various spine pathologies, such as those described herein.

System 10 includes surgical instrument 12, which includes a body 14 extending along an axis L between an end, such as, for example, a proximal end 18 and an opposite end, such as, for example, a distal end 20, as shown in FIGS. 1-3. End 18 includes a proximal most surface 18a and end 20 includes a distal most surface 20a. End 18 includes a gripping surface 28 configured to facilitate manipulation and/or maneuvering of surgical instrument 12. Gripping surface 28 comprises a spherical configuration, such as, for example, a ball handle 22. Ball handle 22 includes an outer circumferential surface having a substantially uniform diameter thereabout and opposing planar portions to enhance gripping. In some embodiments, all or only a portion of surface 28 includes a spherical configuration.

Handle 22 includes an inner surface 23 that defines a cavity, such as, for example, a mating surface 32. Mating surface 32 is configured for disposal of an instrument and/or tool extension, such as, for example, a mating surface of a driver handle 26, as discussed herein. Mating surface 32 is centrally positioned with respect to handle 22. Mating surface 32 is coaxial with axis L. In some embodiments, mating surface 32 may have various cross-section configurations, such as, for example, oval, oblong, triangular, rectangular, square, polygonal, irregular, uniform, non-uniform, variable, tubular and/or tapered. In some embodiments, inner surface 23 may have various surface configurations, such as, for example, smooth and/or surface configurations to enhance engagement with the mating surface of driver handle 26, such as, for example, rough, arcuate, undulating, porous, semi-porous, dimpled, polished and/or textured.

End 18 includes an impact surface 40 configured to receive application of a force to handle 22 with an impact instrument (not shown), such as, for example, a mallet or hammer. Impact surface 40 comprises a cap 42 mounted with end 18 and configured to receive an impacting force. Cap 42 includes a planar surface and a beveled outer circumference, configured to receive an impact force. In some embodiments, impact surface 40 may have various surface configurations, such as, for example, rough, arcuate, dimpled, polished and/or textured to enhance engagement with an impact instrument. In one embodiment, cap 42 is metallic. The impact instrument is engageable with impact surface 40 to create and/or tap a starter hole and/or a pilot hole, as discussed herein, by transferring the force to a bone fastener fixed with surgical instrument 12 to create and/or tap a starter hole and/or a pilot hole with tissue. In one embodiment, impact surface 40 includes mating surface 32 such that impact surface 40 is engageable with a mating surface, such as, for example, an extension 36 of driver handle 26 to rotate, maneuver and/or manipulate surgical instrument 12, as discussed herein.

Body 14 includes an outer surface 15 extending between end 18 and end 20. Surface 15 defines a threaded portion 17 configured for engagement with a sleeve 44, as discussed herein. In one embodiment, surface 15 may have various surface configurations, such as, for example, smooth and/or surface configurations to enhance engagement with sleeve 44, such as, for example, rough, arcuate, undulating, porous, semi-porous, dimpled, polished and/or textured.

Surface 15 includes a plurality of spaced apart ridges 86 that each extend parallel to axis L. Ridges 86 are configured to facilitate gripping by a medical practitioner. Ridges 86 are also configured to facilitate rotation, maneuvering and/or manipulation of body 14. In some embodiments, ridges 86 may be disposed at alternate orientations, relative to axis L, such as, for example, transverse, perpendicular and/or other angular orientations such as acute or obtuse and/or may be offset or staggered. In some embodiments, ridges 86 may have various surface configurations to enhance gripping by a medical practitioner, such as, for example, smooth, rough, arcuate, undulating, porous, semi-porous, dimpled, polished and/or textured.

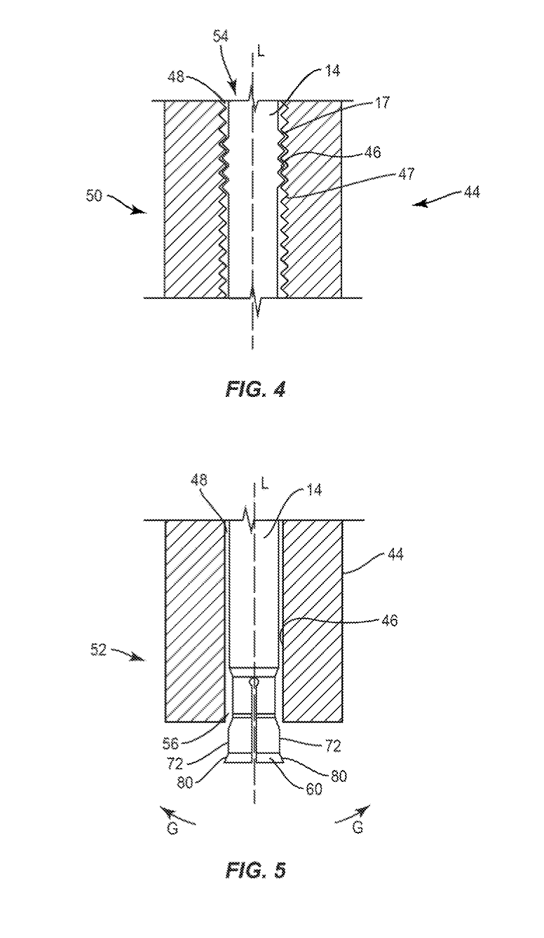

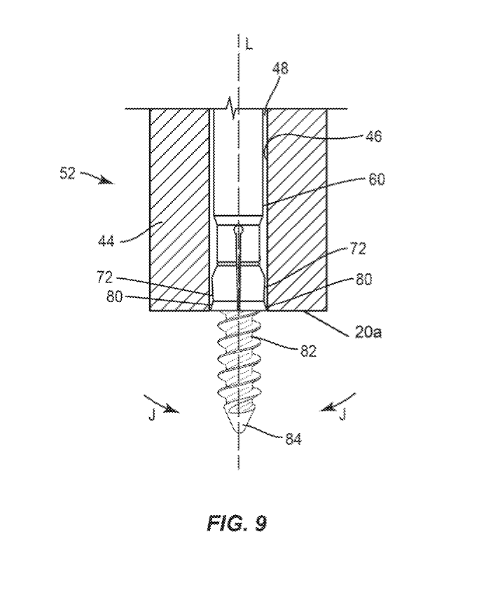

End 20 is configured for engagement with an implant, such as, for example, a bone fastener 82, as shown in FIG. 9. Sleeve 44 is configured to engage and disengage a collet 60 from bone fastener 82, as discussed herein, for releasable fixation with bone fastener 82. Sleeve 44 extends along a portion of body 14 and is configured for axial translation relative to body 14. Sleeve 44 includes an inner surface 46 defining a passageway 48 configured for moveable disposal of body 14, as shown in FIG. 4. In one embodiment, at least a portion of inner surface 46 includes a threaded section 47 configured to rotatably engage threaded portion 17 for axial translation of sleeve 44 relative to body 14, which causes engagement and disengagement of collet 60 with bone fastener 82, as discussed herein. In one embodiment, inner surface 46 may have various surface configurations to enhance engagement of body 14 and/or collet 60, such as, for example, rough, arcuate, undulating, porous, semi-porous, dimpled, polished and/or textured.

Passageway 48 is coaxial with axis L. Sleeve 44 includes a portion 50 and a portion 52, as shown in FIGS. 4 and 5. Portion 50 includes threaded section 47 configured for engagement with threaded portion 17, as described herein, and portion 52 is configured for engagement with collet 60 for releasable fixation with bone fastener 82, as described herein. As shown in FIG. 4, portion 50 includes an opening 54 in communication with passageway 48. Engagement of threaded section 47 and threaded portion 17 facilitates translation of sleeve 44 along body 14 for translation between a first configuration and a second configuration of surgical instrument 12 for releasable fixation with bone fastener 82 and for applying an axial force and/or a torsional force thereto, as discussed herein.

As shown in FIG. 5, portion 52 includes an opening 56 in communication with passageway 48. Surface 46 is configured to engage an outer surface of collet 60 to facilitate expansion to the first configuration and contraction to the second configuration of collet 60, as discussed herein. In some embodiments, portion 50, portion 52, opening 54 and/or opening 56 may have various cross section configurations, such as, for example, cylindrical, oval, oblong, triangular, rectangular, square, hexagonal, polygonal, irregular, uniform, non-uniform, variable and/or tapered.

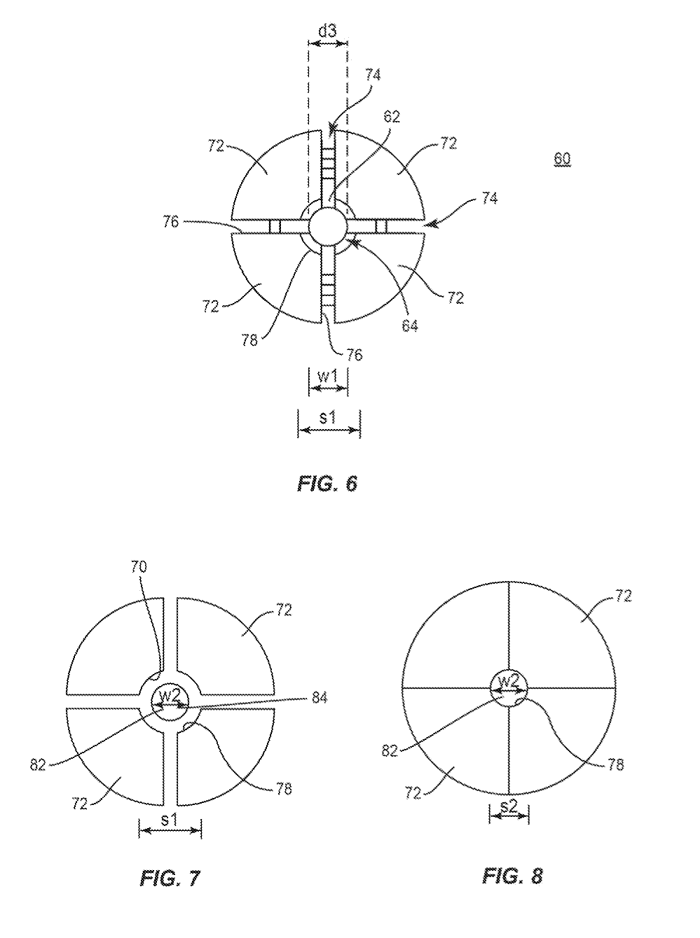

Collet 60 extends from end 20 and is configured for engagement with sleeve 44 for movement between the first configuration and the second configuration. Collet 60 comprises an inner surface 62 defining a passageway 64, as shown in FIGS. 6-8. Passageway 64 is coaxial with passageway 48. Passageway 64 has a cylindrical cross-section configuration and has a uniform diameter d3 along the entire length of passageway 64. In some embodiments, passageway 64 may have various cross section configurations, such as, for example, oval, oblong, triangular, rectangular, square, polygonal, irregular, uniform, non-uniform, variable, tubular and/or tapered.

Collet 60 includes a locking surface 70 defined by a plurality of cantilevered fingers 72 extending radially outward in a tapered configuration. Fingers 72 are circumferentially disposed and are equidistantly spaced apart. Fingers 72 are spaced apart by a gap 74 defined by opposite planar sidewalls 76. Sidewalls 76 of a respective finger 72 converge at a concave portion 78 as sleeve 44 translates over collet 60.

As sleeve 44 axially translates, in the direction shown in by arrow E in FIG. 1, fingers 72 are resiliently biased to deflect outwardly such that fingers 72 are moveable to the first, expanded orientation in which flared portions 80 of each finger 72 are spaced apart, in the direction shown by arrows G in FIG. 5, such that a distance s1 between opposite concave portions 78 is greater than a width w1 of passageway 64. As sleeve 44 axially translates, in the direction shown by arrow F, fingers 72 are driven inwardly by the force of sleeve 44 engaging collet 60 such that fingers 72 are moveable to the second, collapsed or contracted configuration, so that flared portions 80 move, in the direction shown by arrows J in FIG. 9, to capture bone fastener 82 with locking surface 70 and a distance s2 between opposite concave portions 78 is substantially equivalent to width w1.

To capture bone fastener 82 with collet 60, bone fastener 82 is positioned within passageway 64. In the first, expanded orientation of surgical instrument 12, bone fastener 82 has a cylindrical cross sectional configuration with a width w2 that is slightly less than width w1 such that bone fastener 82 is translatable within passageway 64. Distance s1 is greater than width w2, as shown in FIG. 7.

In use, surgical instrument 12 is disposable in the first, non-locking orientation in which flared portions 80 are spaced and bone fastener 82 is translatable within passageway 64, as described herein, by rotating sleeve 44, in the direction shown by arrow HH in FIG. 1, relative to body 14 such that threaded section 47 engages threaded portion 17 and sleeve 44 axially translates relative to body 14, as shown by arrow E in FIG. 1. Surgical instrument 12 is disposable in the second, locking orientation, as shown in FIGS. 8 and 9, by rotating sleeve 44, in the direction shown by arrow H in FIG. 1, relative to body 14 such that threaded section 47 engages threaded portion 17 and sleeve 44 axially translates relative to body 14, as shown by arrow F in FIG. 1. Sleeve 44 axially translates and engages collet 60 such that flared portions 80 engage bone fastener 82 to releasably fix bone fastener 82 with surgical instrument 12 for applying an axial force and/or a torsional force thereto, as described herein. Surgical instrument 12 is disposable in the first, non-locking orientation, as described herein, to release bone fastener 82 from collet 60.

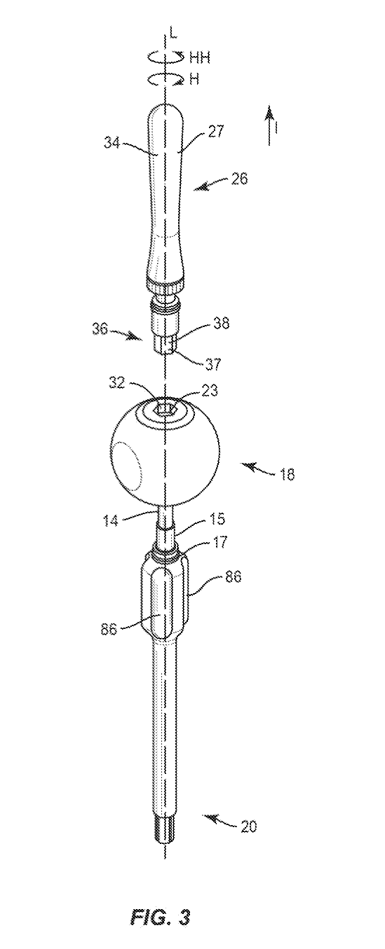

In some embodiments, end 18 comprises driver handle 26 that includes a handle 27 having a handle surface 34 configured for manipulation, maneuvering and/or rotation of driver handle 26, as shown in FIG. 3. In some embodiments, driver handle 26 may be utilized to facilitate engagement of bone fastener 82 with tissue. Handle surface 34 may have different cross-sections such as square, hexagonal, polygonal, triangular, star or hexalobe. Handle surface 34 may have various surface configurations, such as, for example, smooth, rough, arcuate, undulating, porous, semi-porous, dimpled, polished and/or textured. Driver handle 26 includes an extension 36 including an outer surface 37 that defines a mating surface 38. Mating surface 38 is configured for engagement with mating surface 32 to rotate surgical instrument 12 about axis L. In one embodiment, driver handle 26 comprises a ratchet 27 configured for selective and/or indexed rotation of surgical instrument 12 about axis L. In some embodiments, mating surface 38 includes a square, polygonal, triangular, star or hexalobe cross-section configuration to engage mating surface 32. In one embodiment, mating surface 32 defines a hexagonal interface and mating surface 38 includes a corresponding hexagonal interface. In some embodiments, mating surface 32 is engageable with one of a plurality of alternately configured driver handles. In some embodiments, end 20 is connected, such as, for example, fixed with and rotatable relative to end 18, as described herein, such that an instrument or actuator, such as, for example, driver handle 26 or a drill cannot be engaged with end 18 to separate end 18 from end 20.

In some embodiments, bone fastener 82 includes a tip 84, as shown in FIG. 9, configured to form a starter hole and/or pilot hole in bone, as will be described. In some embodiments, tip 84 has a sharp point configured to penetrate tissue, such as, for example, cortical or cancellous bone to fix bone fastener 82 with bone. In some embodiments, tip 84 is beveled. In some embodiments, tip 84 is fluted. In some embodiments, at least a portion of tip 84 is threaded. In some embodiments, at least a portion of tip 84 includes a self-tapping thread. In some embodiments, at least a portion of tip 84 is hollow.

In some embodiments, driver handle 26 is attached with surgical instrument 12 such that the assembly of driver handle 26 and surgical instrument 12 extend a total and/or overall distance, such as, for example, a length. In some embodiments, the length includes a range of approximately 20 through 26 centimeters (cm). In some embodiments, the length extends from a proximal most surface of driver handle 26 to surface 20a. In one embodiment, the length from the proximal most surface of driver handle 26 to surface 20a is equal to or less than approximately 22.5 cm and tip 84 extends at least 3 cm distally from surface 20a in the second, locking orientation of surgical instrument 12, as disabled herein. This configuration includes tactile indicia and/or feel of engagement with an anatomy, as described herein. In some embodiments, a total and/or overall length of surgical instrument 12 extends from surface 18a to surface 20a.

In assembly, operation and use, a surgical implant system, similar to system 10 described herein, is employed with a surgical procedure for treatment of a spinal disorder affecting a section of a spine of a patient, as discussed herein. For example, system 10 can be used with a surgical procedure for treatment of a condition or injury of an affected section of the spine including vertebrae. In some embodiments, one or all of the components of system 10 can be delivered as a pre-assembled device or can be assembled in situ. System 10 may be completely or partially revised, removed or replaced.

For example, system 10 can be employed with a surgical treatment of an applicable condition or injury of an affected section of a spinal column and adjacent areas within a body, such as, for example, vertebrae (not shown). In some embodiments, system 10 may be employed with one or a plurality of vertebra. To treat a selected section of the vertebrae, a medical practitioner obtains access to a surgical site including the vertebrae in any appropriate manner, such as through incision and retraction of tissues. In some embodiments, system 10 can be used in any existing surgical method or technique including open surgery, mini-open surgery, minimally invasive surgery including percutaneous surgical implantation, whereby the vertebrae are accessed through a mini-incision, or sleeve that provides a protected passageway to the area. Once access to the surgical site is obtained, the particular surgical procedure can be performed for treating the spine disorder.

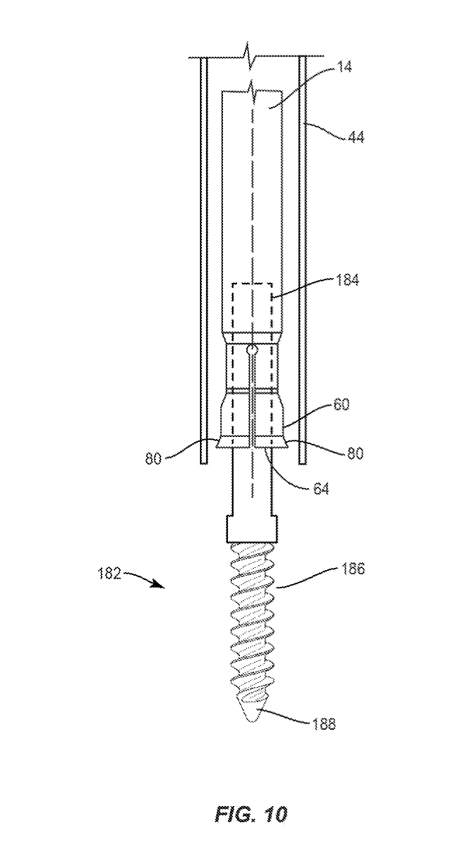

An incision is made in the body of a patient and a cutting instrument (not shown) creates a surgical pathway for delivery of implantable components of system 10 such as, for example, a posted bone screw 182, as shown in FIG. 10 and similar to bone fastener 82 described herein. Bone screw 182 includes a post 184 and a threaded shaft 186. Shaft 186 includes a tip 188. A preparation instrument (not shown) can be employed to prepare tissue surfaces of vertebrae, as well as for aspiration and irrigation of a surgical region.

Surgical instrument 12 is disposable in a first, non-locking orientation, as described herein, by rotating sleeve 44, in the direction shown by arrow HH in FIG. 1, relative to body 14 such that threaded section 47 engages threaded portion 17 and sleeve 44 axially translates relative to body 14, as shown by arrow E in FIG. 1. To capture bone screw 182 with collet 60, post 184 is positioned within passageway 64. Post 184 is translatable along axis L within passageway 64 such that the distance that shaft 186 extends distally beyond collet 60 is selectively adjustable. Surgical instrument 12 is disposable in a second, locking orientation, as described herein, by rotating sleeve 44, in the direction shown by arrow H in FIG. 1, relative to body 14 such that threaded section 47 engages threaded portion 17 and sleeve 44 axially translates relative to body 14, as shown by arrow F in FIG. 1, to releasably fix bone screw 182 with surgical instrument 12.

In the locking orientation, a force, such as, for example, a longitudinal or axial force is applied to impact surface 40, by for example, a mallet or hammer, which is transferred to bone screw 182 such that tip 188 creates a cavity, such as, for example, a starter hole in tissue, such as, for example, bone, as described herein. In one embodiment, tip 188 penetrates cortical bone adjacent a posterior side of a sacrum to form a starter hole by use of a longitudinal pushing force exerted on handle 22.

With the starter hole created in bone, a force, such as, for example, a substantially longitudinal or axial force is applied to impact surface 40, by for example, a mallet or hammer, which is transferred to bone screw 182 such that tip 188 creates a pilot hole in the bone from the starter hole. The depth of the pilot hole may be increased by driving body 14, in the direction shown by arrow F in FIG. 1 by, for example, impacting impact surface 40.

In some embodiments, surgical instrument 12 facilitates confirmation of a selected trajectory of bone screw 182 with tissue, such as, for example, pedicle walls of vertebrae. For example, a medical practitioner may remove bone screw 182 from the pilot hole and apply tactile pressure with, for example, a hand or fingers, to the tissue adjacent and/or surrounding the pilot hole to ensure accurate trajectory of bone screw 182 within the pedicle walls. Upon confirmation of the selected trajectory of bone screw 182, bone screw 182 is re-inserted into the pilot hole. In some embodiments, the depth of the pilot hole may be increased by use of driver handle 26. Driver handle 26 is attached with handle 22 by engaging mating surface 32 with mating surface 38.

Driver handle 26 is rotated, in the direction shown by arrow H in FIG. 1, to apply a torsional force to bone screw 182 and increase the depth of the pilot hole and/or fasten bone screw 182 with tissue. As the depth of the pilot hole increases, shaft 186 engages the outer layer of cortical bone such that further rotation of bone screw 182 about axis L causes tip 188 to move through the plot hole and the outer layer of cortical bone and into a layer of cancellous bone. In some embodiments, bone screw 182 is rotated until the shaft of bone screw 182 penetrates the vertebra to fix bone screw 182 with the tissue. This configuration implants and fixes bone screw 182 with bone in a single step to facilitate a procedural flow of a surgical method and streamline the surgical method.

In some embodiments, surgical instrument 12 is delivered through a surgical pathway to a location adjacent vertebrae at a surgical site such that tip 188 penetrates an outer layer of cortical bone of vertebrae, for example, a posterior side of the vertebrae to create a starter hole and/or a pilot hole. The components of system 10, including surgical instrument 12 and bone screw 182 are employed to augment one or more surgical treatments. Surgical instrument 12 is disposable in the first, non-locking orientation, as described herein, to release bone screw 182 from collet 60.

Surgical instrument 12 may be re-assembled for use in a surgical procedure. In some embodiments, surgical instrument 12 may comprise various instruments including a lock and collet configuration of the present disclosure, with, for example, inserters, extenders, reducers, spreaders, distractors, blades, retractors, clamps, forceps, elevators and drills, which may be alternately sized and dimensioned, and arranged as a kit.

Upon completion of a procedure, surgical instrument 12, surgical instruments and/or tools, assemblies and non-implanted components of system 10 are removed and the incision(s) are closed. One or more of the components of system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of system 10. In some embodiments, system 10 may include one or a plurality of plates, connectors and/or bone fasteners for use with a single vertebral level or a plurality of vertebral levels.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument comprising:
a first end including a gripping surface, at least a portion of the gripping surface having a spherical configuration; and
a second end connected with the first end such that an instrument is prevented from engaging the first end to separate the first end from the second end, the second end being configured to engage a bone fastener,
wherein a distance from the first end to the second end is equal to or less than approximately 22.5 cm.

2. A surgical instrument as recited in claim 1, wherein the second end engages the bone fastener such that the bone fastener probes, taps and forms a threaded connection of the bone fastener with tissue.

3. A surgical instrument as recited in claim 1, wherein the first end comprises a sphere.

4. A surgical instrument as recited in claim 1, wherein the first end comprises a ball shaped handle.

5. A surgical instrument as recited in claim 1, wherein the first end comprises a mating surface engageable with a mating surface of a driver handle.

6. A surgical instrument as recited in claim 5, wherein the mating surfaces comprise a hexagonal configured interface.

7. A surgical instrument as recited in claim 5, wherein the first end includes an impact surface that includes the mating surface of the first end.

8. A surgical instrument as recited in claim 7, wherein the impact surface comprises a cap mounted with the first end.

9. A surgical instrument as recited in claim 7, wherein the impact surface includes a planar surface.

10. A surgical instrument as recited in claim 7, wherein the impact surface is axially engageable to tap the bone fastener with tissue and the first end is rotatable to form a threaded connection of the bone fastener with tissue.

11. A surgical instrument as recited in claim 1, wherein the first end comprises a mating surface engageable with a mating surface of a driver handle, the mating surface of the first end being engageable with one of a plurality of alternately configured driver handles.

12. A surgical instrument as recited in claim 1, wherein the first end comprises a mating surface engageable with a mating surface of a driver handle, the driver handle comprising a ratchet.

13. A surgical instrument as recited in claim 1, wherein the second end includes a collet for capturing the bone fastener.

14. A surgical instrument as recited in claim 1, wherein the second end includes a sleeve configured to capture a posted bone fastener.

15. A method for treating a spine, the method comprising the steps of:
providing a surgical instrument comprising a first end including a drive handle and a gripping surface, and a second end connected with the first end such that an instrument is prevented from engaging the first end to separate the first end from the second end;
connecting a bone fastener to the second end;
applying a first force to the surgical instrument such that the first force is applied to the bone fastener to form a cavity in tissue;
applying a second force to the surgical instrument such that the second force is applied to the bone fastener to form a pilot hole from the cavity; and
rotating the drive handle to fasten the bone fastener with the tissue.

16. A method as recited in claim 15, further comprising the step of confirming trajectory of the pilot hole, which includes removing the bone fastener from the pilot hole and applying tactile contact to tissue adjacent the pilot hole.

17. A method as recited in claim 16, further comprising the step of re-inserting the bone fastener with the pilot hole.

18. A method as recited in claim 16, wherein the gripping surface comprises a sphere.

19. A method as recited in claim 15, wherein the driver handle comprises a ratchet.

20. A surgical instrument comprising:
a first end including a gripping surface, at least a portion of the gripping surface having a spherical configuration; and
a second end connected with the first end such that an instrument is prevented from engaging the first end to separate the first end from the second end, the second end being configured to engage a bone fastener, wherein a distance from the first end to the second end is equal to or less than approximately 22.5 cm,
wherein the surgical instrument is disposable between a first configuration such that the first end is rotatable relative to the second end and a second configuration such that the second end is configured to apply a torsional force.
